(19) United States
(12) Patent Application Publication
(10) Pub. No.: US 2003/0022174 A1
(43) Pub. Date: Jan. 30, 2003

(54) Cancer diagnostic method using P40 subunit of EIF3

(76) Inventors: Tapio Visakorpi, Tampere (FI); Jorma Isola, Pirkkala (FI); Nina Nupponen, Bethesda, MD (US); Volodymyr Ovod, Tampere (FI)

Correspondence Address: NIXON & VANDERHYE P.C., 8TH Floor, 1100 North Glebe Road, Arlington, VA, 22201, US

(21) Appl. No.: 09/878,328

(22) Filed: Jun. 12, 2001

Related U.S. Application Data
(63) Continuation of application No. PCT/FI99/01039, filed on Dec. 15, 1999.

(30) Foreign Application Priority Data
Dec. 16, 1998 (FI) 982722

(51) Int. Cl. C12Q 1/68; G01N 33/574; C12P 19/34

(52) U.S. Cl. 435/6; 435/7.23; 435/91.2

(57) ABSTRACT

The present invention relates to a novel diagnostic method for detecting progression of cancer. In particular, the present invention relates to a diagnostic method for detecting and identifying aggressive forms of certain carcinomas, especially breast cancer and prostate cancer. The present invention also relates to the use of p40 subunit of eukaryotic translation initiation factor 3 (eIF3-p40) or a variant or fragment thereof as a diagnostic agent or in therapy.

### FIELD OF THE INVENTION

The present invention relates to a novel diagnostic method for detecting progression of cancer, particularly for detecting and identifying aggressive forms of certain carcinomas, especially breast cancer and prostate cancer. The present invention also relates to the use of p40 subunit of eukaryotic translation initiation factor 3 (eIF3-p40) or a variant or a fragment thereof as a diagnostic agent or in therapy.

### BACKGROUND OF THE INVENTION

The incidence of breast and prostate carcinomas is steadily increasing. Simultaneously, methods for diagnosis and therapy have improved. Fortunately, mainly due to early diagnosis and effective treatment of these diseases the mortality rates have not risen as much as the incidence rates. Breast cancer is the most common cancer in women, whereas prostate cancer is the most common cancer among men in industrialized countries.

Because of the wide variability in the course of the disease, it is difficult to assess the prognosis and to select an optimal treatment for individual patients, especially for those suffering from aggressively proliferating forms of cancer. Therefore, various means have been developed for identifying the prognostic clinicopathological factors, which can be associated with aggressive forms of carcinomas.

Generally in clinical practice, the prognosis of the disease and the selection of the post-operational treatment of cancer are currently mainly based on the evaluation of the clinical stage of the disease and the histological, especially nuclear, gradus of the tumors. These methods do not, however, predict well the progression rate of the disease. The determination of steroid hormone receptor content is additionally used in the prognosis of the disease and the selection of adjuvant chemotherapy in breast cancer. Other means are also used experimentally to supplement the evaluation of treatment recommendations. They include the determination of the growth rate of the cancer, DNA flow cytometric analysis, and immunohistochemical analysis of prognostic markers, such as various oncogenes, for example erbb-2 oncogene, oncogenic products and tumor suppressor genes.

Likewise in prostate cancer, staging of the tumor and grading of the tumor on the basis of, for example, glandular differentiation or nuclear anaplasia, are widely used in the clinical routine to help the determination of the prognosis and the treatment. The analysis of the DNA content by means of flow cytometry and the analysis of cell proliferation activity as well as growth factors, oncogenes and tumor suppressor genes may also be used.

None of these methods, however, fulfills the requirements for accurate prognostic evaluation. They are also expensive and require special equipment, reagents and skills to perform.

Thus, additional means for identifying especially high-risk patients in terms of recurrence of the cancer are still needed. Especially in node-negative breast cancer, novel and reliable means are urgently needed for evaluating the prognosis of the disease and for selecting the treatment, in particular for selecting adjuvant chemotherapy. In prostate cancer, the most critical question is predicting the risk of tumor recurrence after prostatechtomy.

The development and progression of cancer is considered to be induced by multiple genetic alterations, such as gene amplification. Several amplified oncogenes have been identified in cancer (Alitalo, K. and Schwab, M., Adv Cancer Res, 47, 235-81, 1986; Brison, O., Biochim Biophys Acta, 1155, 25-41, 1993), but studies by comparative genomic hybridization (CGH) (Kallioniemi et al., Science, 258, 818-21, 1992) have recently indicated that known oncogenes account for only a part of the detected amplifications in human neoplasias (Forozan et al., Trends Genet, 13, 405-9, 1997). The long arm of chromosome 8 (8q) is one of the most common regions of amplification in cancers of several organs, such as bladder and ovarian cancer, but especially carcinomas in the breast and the prostate (Visakorpi et al., Cancer Res, 55, 342-7, 1995; Cher et al., Cancer Res, 56, 3091-102, 1996; Nupponen et al., Am J Pathol, 153, 141-8, 1998; Tirkkonen et al., Genes Chromosomes Cancer, 21, 177-84, 1998). CGH studies have verified that almost 80% of locally recurring hormone-refractory prostate carcinomas and distant metastases contain an 8q gain, whereas it is present only in about 5% of primary untreated prostate carcinomas (Visakorpi et al., supra; Cher et al., supra; Nupponen et al., supra). Almost half of primary breast carcinomas display a copy number gain at 8q (Tirkkonen et al., supra). In both cancer types, the gain of 8q is also associated with the aggressive phenotype of the disease (Isola et al., Am J Pathol, 147, 905-11, 1995; Van den Berg et al., Clin Cancer Res, 1, 11-18, 1995). In breast cancer, the 8q gain has been shown to be associated with poor prognosis of patients (Isola et al., supra), whereas in prostate cancer the amplification of 8q24 region occurs with the presence of lymph-node metastases (Van den Berg et al., supra). However, any specific relationship between an identified potential target gene in 8q and certain specific forms of cancer has not been shown. The identification of such a relationship would lead to urgently desired improvements in the field of cancer diagnostic and would be beneficial to cancer patients.

Two independently amplified sub-regions, 8q21 and 8q23-q24, have been identified within the 8q arm (Cher et al., supra; Nupponen et al., supra), suggesting the presence of several target genes for the amplification. These two minimal commonly amplified regions comprise together a chromosomal fragment of almost 60 Mb containing possibly more than a thousand genes. U.S. Pat. No. 5,658,730 takes advantage of the overall amplification of 8q24 and discloses a diagnostic method for prostate cancer progression, the method determining the presence of an amplified 8q24.1 -24.2 chromosome band segment. However, no connection is disclosed between the amplification of any specific gene of this chromosome area and prostate cancer.

### SUMMARY OF THE INVENTION

We have now discovered that the p40 subunit of eukaryotic translation initiation factor 3 (eIF3) gene, located at 8q23, is amplified and over-expressed in a large fraction of breast and prostate cancer implicating that it is a putative target gene for the 8q amplification. This discovery provides a novel means which can be utilized in the development and improvement of cancer diagnostics. The p40 subunit of eukaryotic translation initiation factor 3 (eIF3) gene may also find use in the therapy of carcinomas.

An object of the invention is thus to provide a diagnostic method that is useful in identifying and detecting aggressive forms of carcinoma, especially of breast cancer or prostate cancer, in biological samples.

Another object of the invention is to provide a reliable method that is useful in the prognosis of an optimal treatment for patients suffering from carcinoma, especially from an aggressive form of carcinoma, especially of breast cancer or prostate cancer.

Yet another object of the invention is to provide means that can be used for the treatment of carcinomas, especially aggressive forms of carcinomas, in particular of breast cancer or prostate cancer.

The present invention relates to a new method for diagnosing aggressive forms of carcinomas, especially those of breast and prostate cancer, by detecting the presence or absence of amplification and/or expression of p40 subunit of eukaryotic translation initiation factor 3 (eIF3-p40) or a functional variant or functional fragment thereof in a biological sample.

The present invention also relates to a use of p40 subunit of eukaryotic translation initiation factor 3 or a functional variant or functional fragment thereof for the diagnosis of aggressive forms of carcinomas, especially of breast and prostate cancer.

The present invention further relates to a method of identifying of cancer patients, especially those suffering from an aggressive form of cancer, such as breast and prostate cancer, who need and can be helped by adjuvant chemotherapy as well as to a method of predicting an optimal treatment for such patients, by detecting the presence or absence of amplification and/or expression of p40 subunit of eukaryotic translation initiation factor 3 or a functional variant or functional fragment thereof in a biological sample obtained from said patients.

The present invention also relates to a use of p40 subunit of eukaryotic translation initiation factor 3 or a functional variant or functional fragment thereof for the identification of patients suffering from of aggressive forms of carcinomas, such as breast cancer and prostate cancer.

The present invention also relates to a method for evaluating of the aggressivity of carcinomas by determining the presence or absence of p40 subunit of eukaryotic translation initiation factor 3 or a functional variant or functional fragment thereof in a tumor sample.

The present invention also relates to a use of p40 subunit of eukaryotic translation initiation factor 3 or a functional variant or functional fragment thereof as a diagnostic agent and to a diagnostic kit containing said subunit or a variant or a fragment thereof as one of the means for detecting proliferating diseases, such as cancer, in particular breast or prostate cancer.

The present invention also relates to a use of p40 subunit of eukaryotic translation initiation factor 3 or a functional variant or fragment thereof or a mutated variant of fragment thereof in therapy of proliferating diseases, such as cancer, in particular breast or prostate cancer.

The present invention further relates to diagnostic kits containing reagents, such as antibodies, to detect p40 subunit of eukaryotic translation initiation factor 3.

The present invention further relates to antibodies that are capable of identifying p40 subunit of eukaryotic translation initiation factor 3 and to cell lines capable of producing antibodies against p40 subunit of eukaryotic translation initiation factor 3.

### BRIEF DESCRIPTION OF THE DRAWINGS

FIG. 1 shows a two-color fluorescence in situ hybridization (FISH) analysis demonstrating a high-level amplification of eIF3-p40 (green signals) in (A) breast cancer cell line SK-Br-3, (C) a primary breast carcinoma, (D) prostate cancer cell line PC-3, and (E) a hormone-refractory prostate carcinoma. The eIF3-p40 gene is present as several copies in two large marker chromosomes (shown by the arrows), as well as in several smaller chromosomes in SK-Br-3. There is only one chromosome 8 centromere signal (red signal) in SK-Br-3. interphase nuclei of cell line PC-3 and uncultured breast and prostate tumors show multiple copies of eIF3-p40, and only two copies of centromere 8. FIG. 1B shows the location of eIF3-p40 (green signals) in normal human chromosome 8 in 8q23 approximately 12 Mb centromeric of c-myc (red signals).

FIG. 2 shows the results of a Northern blot analysis demonstrating increased expression of eIF33-p40 in breast cancer cell lines MDA436, MCF-7, and SK-Br-3, and in prostate cancer cell line PC-3, as compared to the expression level in breast cancer cell line ZR75-1 and in prostate cell lines DU145 and LNCaP. The relative level of expression of the genes is given in proportion to the expression in ZR75-1. The expression of β-actin is used to control the loading differences. The relative copy number (gene vs. centromere copy number) of eIF3-p40 and c-myc are also shown.

FIG. 3 shows the results of eIF3-p40 mRNA in situ hybridization demonstrating over-expression in (A) a hormone-refractory prostate carcinoma and in (B) a primary breast carcinoma, and (C) low-level expression in benign prostate hyperplasia and (D) in a primary breast carcinoma without eIF4p40amplification. (A) and (B) correspond to the FISH images in FIGS. 1C and 1E, respectively. Hybridization signals were visualized with an epipolarization filter (magnification×400).

FIG. 4 shows the mean (±SEM) level of the eIF3-p40 expression in (A) benign prostate hyperplasia (BPH; n=9) and recurrent hormone-refractory prostate cancer (n=27), as well as (B) in primary breast carcinomas (n=34). The mRNA in situ hybridization signals were quantitated from an autoradiograph film by means of Personal Densitometer SI (Molecular Dynamics Inc.) in terms of pixel intensity.

FIG. 5 shows the mean copy numbers of eIF3-p40 and c-myc in 5 breast tumors. Tumors 1, 2 and 3 (T1, T2 and T3. respectively) from selected breast cancer material show clearly a higher copy number of eIF3-p40 than c-myc. However, one tumor (T4) from the selected breast cancer material and another tumor (T5) from the unselected material display more c-myc than eIF3-p40 signals.

FIG. 6 is a Western blot showing the specific reactivity of the mouse anti-eIF3-p40 monoclonal antibodies P40 6.1 and P40 4.1 against native proteins from the epithelial cell line SK-Br-3. The samples were run on PAGE, transferred to a nitrocellulose filter and probed with sera as follows: Lane A monoclonal antibody P40 6.1, (B) monoclonal antibody P40 4.1, and (C) no primary antibody. Both primary antibodies recognize a 40 kDa band corresponding to the size of the eIF3-p40 protein.

### DETAILED DESCRIPTION OF THE INVENTION

The present invention is based on studies aiming for the identification of over-expressed target genes for the 8q amplification. Suppression subtractive hybridization (SSH) (Diatchenko et al., Proc Natl Acad Sci USA, 93, 6025-30, 1996) was applied to identify over-expressed transcripts in breast cancer cell line SK-Br-3, which shows high-level amplification at 8q13-q21.3 and 8q23-q24.1 by comparative genomic hybridization (CGH) (Kallioniemi et al, supra). Breast cancer cell line ZR75-1 showing normal relative copy numbers at 8q was used as a reference. cDNAs from SK-Br-3 were subtracted against those from ZR-75-1 and the resulting cDNAs were cloned into pCR2.1 vector. Random clones were picked from the subtracted library and then sequenced.

Database searches with BLASTN revealed that the first redundant clone (named A8) recognized an EST clone 595376 (accession no. AA173710), which according to the Unigene database was located in the region of interest, between markers D8S276 (8q22.3) and D8S1799 (8q24).

Next, it was verified by a Northern blot analysis that A8 was differentially expressed in SK-Br-3 and ZR-75-1. The database search also showed that the sequence of A8 was identical to a recently cloned gene, eukaryotic translation initiation factor 3 subunit p40 (eIF3-p40) (Asano et al., J Biol Chem, 272, 27042-27052, 1997). To map the gene precisely, the genomic clone for eIF3-p40 was obtained by screening a human PAC library with a polymerase chain reaction (PCR) and specific primers designed for the gene. Using fluorescence in situ hybridization (FISH), eIF3-p40 was localized to 8q23, about 12 Mb centromeric from c-myc.

To show that eIF3-p40 is amplified in carcinomas, especially in breast and prostate carcinomas, the eIF3-p40 gene copy number status in breast and prostate cancer was first studied by analyzing four breast cancer cell lines, SK-Br-3, MDA436, MCF-7, and ZR-75-1, and three prostate cancer cell lines, PC-3, DU-145, and LNCaP, by FISH. High-level amplification (five or more copies of the gene or an eIF3-p40/centromere ratio >2) of eIF3-p40 was found in SK-Br-3, MDA-436, MCF-7₁ and PC-3, in accordance with the gain of 8q found by CGH in these cell lines (FIG. 2).

Additionally, hormone-refractory locally recurrent prostate carcinomas and untreated primary breast carcinomas were screened for the eIF3-p40 copy number by FISH. 30% of the prostate carcinomas showed high-level amplification of eIF3-p40, whereas the remaining cases showed a low-level copy number gain (3 to 4 copies) of eIF3-p40. In prostate tumors with high-level amplification, the mean (±SD) copy number of eIF3-p40 was 6.7 (±1.5). 18% of the breast cancers showed high-level amplification of eIF3-p40, 43% showed a low-level gain, while the remaining tumors (39%) showed two copies of the eIF3-p40. The mean copy number of eIF3-p40 was 8.5 (±2.9) in the breast tumors with high-level gene amplification.

Also, 19 selected breast carcinomas with high-level c-myc amplification demonstrated by Southern blot (Borg et al., Int J Cancer, 9, 687-91, 1992) were analyzed. All tumors tested displayed high-level amplification of eIF3-p40 with a mean copy number of 21.8 (±21.12).

Since eIF3-p40 is localized close to the c-myc oncogene, the copy number of c-myc was also studied by FISH. In the breast and prostate cancer cell lines, the copy numbers of c-myc and eIF3-p40 were identical, except in PC-3, where eIF3-p40 was present in 15 copies and c-myc only in nine copies. All unselected hormone-refractory prostate carcinomas showed equal copy numbers of eIF3-p40 and c-myc, whereas one of the unselected breast carcinomas showed high-level amplification of c-myc, but only low-level amplification of eIF3-p40. Of the 19 selected breast carcinomas tested, three showed about five times higher copy number of eIF3-p40 than c-myc, whereas one case showed about twice as many c-myc signals as eIF3-p40 signals.

The amplification of the putative target proto-oncogenes is thought to lead to their over-expression. The expression levels of eIF3-p40 and c-myc in cancer cell lines were compared using Northern analysis. There was no clear association between the expression and the amplification status of c-myc, whereas the expression of eIF3-p40 was related to its gene copy number.

The expression level of eIF3-p40 was examined in prostate and breast tumors with semi-quantitative mRNA in situ hybridization. The hormone-refractory prostate carcinomas expressed over four times more eIF3-p40 than benign prostate hyperplasia tissues (FIG. 4) (Mann-Whitney U-test; p=0.0021). The level of eIF3-p40 expression was higher in breast carcinomas with high-level amplification than in breast carcinomas with low-level or no amplification (Kruskal-Wallis test; p=0.028). Thus, over-expression of the eIF3-p40 gene was significantly associated with its amplification, suggesting that it is one of the putative target genes amplified in the 8q23-q24 region.

The amplification of the long arm of chromosome 8 is one of the most common DNA sequence copy number alterations in breast and prostate cancer (Visakorpi et al., supra; Cher et al., supra; Nupponen et al., supra; Tirkkonen et al., supra). The eIF3-p40 gene was identified as a candidate gene for the 8q amplification. The high-level amplification of the gene was found in one third of the hormone-refractory recurrent prostate carcinomas and in about one fifth of the breast carcinomas. This indicates that the amplification of eIF3-p40 is one of the most common types of gene amplification in these tumor types.

eIF3-p40, which was found to be amplified and over-expressed in breast and prostate cancers, has not been implicated in the development or progression of cancer before. It is a subunit of the largest (˜600 kDA) eukaryotic translation initiation factor protein complex, which has a central role in the initiation of translation. The eIF3-complex binds to 40S ribosomal units in the absence of other initiation factors and preserves the dissociated state of 40S and 60S ribosomal subunits. It also stabilizes eIF2.GTP.Met.tRNA binding with 40S and mRNA binding with ribosomes (Hershey et al., Biochimie, 78, 903-907,1996). Very little is known about the eIF3-p40 subunit itself. On the basis of the sequence homology, it seems to be related to mouse protein Mov-34 (Asano et al., J Biol Chem, 272, 27042-27052,1997). The gene product of a human homologue of Mov-34 is a component of the 26S protease.

According to the diagnostic method of the present invention, the presence or absence of the eIF3-p40 gene can be detected from a biological sample by any known detection method suitable for detecting a gene copy number or expression, i.e. methods based on detecting the copy number of the gene (or DNA) and/or those based on detecting the gene expression products (mRNA or protein). Such methods are easily recognized by those skilled in the art and include in situ hybridizations, such as fluorescence in situ hybridization (FISH) and mRNA in situ hybridization, Southern analysis, RT-PCR, Northern and Western analyses, immunohistochemistry, and other immunoassays. Preferable methods are those suitable for use in routine clinical laboratories, such as FISH and immunohistochemistry.

In the diagnostic method of the invention, the biological sample can be any sample containing tumor cells, such as a biopsy sample from the breast, prostate, a lymph node or other tissues containing metastatic lesions, including circulating cancer cells. The biological sample can also be a body fluid, such as whole blood, serum, plasma, urine, lymph, and a cerebrospinal fluid sample. The biological sample can be pretreated, if necessary, in a suitable manner known to those skilled in the art.

The diagnostic kit of the present invention comprises reagents necessary for the detection of eIF3-p40. These reagents include specific antibodies, preferably monoclonal antibodies, capable of identifying eIF3-p40 or its gene products, other antibodies, markers and standards that are needed for visualization or quantification as well as buffers, diluents, washing solutions and the like, commonly contained in a commercial reagent kit. Alternatively, the diagnostic kit of the present invention may comprise eIF3-p40 or its functional variant or fragment together with suitable reagents, such as those listed above, needed for the detection of the antibodies against the eIF3-p40.

In therapy, an altered form of the eIF3-p40 gene or antisense oligonucleotide against the p40 gene can be used therapeutically in any technique presently available for gene therapy to prevent the progression of a proliferating disease. In particular, tumor cell growth may be slowed down or even stopped by such therapy. Such techniques include the ex vivo and in situ therapy methods, the former comprising transducing or transfecting an altered eIF3-p40 gene in a vector or antisense oligonucleotides containing cells to the patient, and the latter comprising inserting the altered gene or oligonucleotide into a carrier, which is then introduced into the patient. Depending on the disease to be treated, a transient cure or a permanent cure may be achieved. Alternatively, poly- or monoclonal antibodies can be used to suppress the function of the eIF3-p40 protein, and thus tumor cell growth may be slowed down or even stopped. Antibodies against p40 could also be used to carry other agents, such as cytotoxic substances, to the cancer cells over-expressing the p40 gene. Such agents could then be used to kill specifically the cancer cells.

The present invention provides a more reliable, rapid and easier diagnosis of various proliferating diseases, such as carcinomas, especially breast and prostate carcinomas, and opens new possibilities in the therapy thereof.

The invention will be elucidated below by the following non-limiting examples. The cell lines and tumors used in the Examples were as follows. Breast cancer cell lines SK-Br-3 (ATTC no. HTB-30), MDA-436 (ATTC no. HTB-130), MCF-7 (ATTC no. HTB-22), and ZR-75-1 (ATTC no. CRL-1500) and prostate cancer cell lines PC-3 (ATTC no. CRL-1435), DU-145 (ATTC no. HTB-81), and LNCaP (ATTC no. CRL-1740), were obtained from the American Type Culture Collection (Rockville, Md., USA) and cultured in the recommended conditions. The tumor material was obtained from the Tampere University Hospital and it consisted of two sets of tumors.

The first set of tumor samples were formalin-fixed paraffin-embedded hormone-refractory prostate carcinomas (n=44) taken from patients after failure to hormonal therapy. All samples were obtained from transurethral resections (TUR), which were done to relieve urethral obstructions. The average time from the diagnosis (the beginning of the hormonal therapy) to the progression was 44 months (range: 8-113 months). The second set of tumor samples consisted of thirty-nine freshly frozen primary invasive breast carcinomas taken from patients prior to any treatment. In addition, 19 breast carcinoma imprint touch preparations were obtained from the Department of Oncology, University of Lund, Sweden. These tumors are known to contain c-myc amplification according to Southern analysis (Borg et al., supra).

### EXAMPLE 1

Identification of eIF3-p40 as a Target Gene of the Amplification

Suppression subtractive hybridization (SSH) was used to identify over-expressed transcripts in breast cancer cell line SK-Br-3, which shows high level amplification at 8q13-q21.3 and 8q23-q24.1 by CGH (Kallioniemi et al., supra). Breast cancer cell line ZR75-1 showing normal relative copy numbers at 8q was used as a reference.

SSH was done with PCR-Select™ cDNA Subtraction Kit (Clontech, Calif., USA) with minor modifications as described by Diatchenko et al. (Proc Natl Acad Sci USA, 93, 6025-30, 1996). Total RNAs were first isolated from breast cancer cell lines SK-Br-3 and ZR75-1 by TRIzol Reagent (Gibco BRL, Grand Island, N.Y., USA), and mRNAs were isolated from these using Dynabeads (Dynal A. S., Oslo, Norway) for use in cDNA synthesis. cDNA from SK-Br-3 was used as a tester and cDNA from ZR75-1 as a driver in the subtraction hybridization. The resulting subtracted cDNAs were subcloned into pCR® 2.1-TOPO vector (Invitrogen, Carlsbad, Calif., USA). The inserts were amplified by PCR using adapter-specific primers (Clontech) from randomly picked clones, and sequenced using ABI PRISM Dye Terminator Cycle Sequencing Ready Reaction kit (Perkin-Elmer Corp, Foster City, Calif., USA) and ABI310 sequencer (Perkin-Elmer).

Database searches with BLASTN revealed that the first redundant clone (named A8) recognized an EST clone 595376 (accession no. AA173710), which according to the Unigene database was located in the region of interest, between markers D8S276 (8q22.3) and D8S1799 (8q24).

To verify that A8 was differentially expressed in SK-Br-3 and ZR-75-1, a Northern blot analysis was performed. Total RNAs from the cell lines were isolated by TRizol Reagent (Gibco BRL). Twenty jig of the total RNA was electrophoresed, transferred to a nylon membrane (Hybond-N, Amersham, Arlington Heights, Ill.), and hybridized sequentially with the α³²P-labelled (Amersham International) probes (Random Primed DNA labeling kit, Boehringer Mannheim) for eIF3-p40 (1.2 kb insert of EST 346021; GenBank accession no. W72146), c-myc (2.2 kb insert of EST 51699; GenBank accession no. H24033), and βactin (Clontech) using standard protocols. The hybridization signals were detected and quantitated with Phosphoimager (Molecular Dynamics Inc., Sunnyvale, Calif., USA) and ImageQuaNT software program (Molecular Dynamics Inc).

A database search also showed that the sequence of A8 was identical to the recently cloned gene, eukaryotic translation initiation factor 3 subunit p40 (eIF3-p40) (Asano et al., J Biol Chem, 272, 27042-27052, 1997).

To map the gene precisely, fluorescence in situ hybridization (FISH) of eIF3-p40 was performed essentially as described earlier (Hyytinen et al., Cytometry, 16, 93-99,1994). The genomic clone for eIF3-p40 was obtained from screening a human PAC library using polymerase chain reaction (PCR) with primers specific to eIF-p40 (Institute of Biotechnology, University of Helsinki). The sequences of the primers used were 5′-GCCCAGGCTCTTCAAGAATAC-3′ (sequence id. no. 1) and 5′ ATAGCCAAAATCGGCAATGA-3′ (sequence id. no. 2). A genomic P1-probe for c-myc was obtained from RMC (RMC08P001, Berkeley, Calif., USA). The probes were labeled with biotin-16-dUTP or digoxigenin-11dUTP (Boehringer Mannheim) using nick-translation. A Texas-Redlabeled chromosome 8 asatellite probe was used as a reference probe (CEP8, Vysis Inc., Downers Grove, Ill.).

The mapping of eIF3-p40 (green signals in FIG. 1B) to normal human chromosome 8 indicates that it is located in 8q23, approximately 12 Mb centromeric of the c-myc (red signals in FIG. 1B). FLpter values, which were used to estimate the localization of eIF3-p40, were measured for both eIF3-p40 (mean FLpter value 0.8096) and c-myc (mean FLpter value 0.8894) using Scilimage software program (TNO, Delft, the Netherlands).

### EXAMPLE 2

Amplification of eIF3-p40 in Both Breast Cancer and Prostate Cancer Cell Lines

To show that eIF3-p40 is amplified in both breast and prostate cancer, the p40 gene copy number status in breast and prostate cancer was first determined by analyzing four breast cancer cell lines, SK-Br-3, MDA436, MCF-7, and ZR-75-1, and three prostate cancer cell lines, PC-3, DU-145, and LNCaP, by FISH. Metaphase and interphase cell preparations from the cancer cell lines and normal blood lymphocytes, nuclei from paraffin-embedded prostate carcinomas, and frozen breast carcinomas were used for the FISH analysis. Metaphase and interphase FISH was performed as described in details elsewhere (Hyytinen et al., Cytometry, 16, 93-99, 1994). Before the hybridization, prostate cancer samples were pretreated by heating in 59% glycerol/0, 1×standard saline citrate (SSC, pH 7.5) solution at 90° C. for 3 minutes to improve hybridization efficiency.

Slides were denatured in a 70% formamide-2×SSC solution at 73° C. for 3 minutes. Signal copy numbers were counted from 100 randomly chosen individual nuclei. Control hybridizations included normal lymphocytes and formalin-fixed paraffin-embedded benign prostate hyperplasia (BPH) samples (n=10). The probes used recognized a single copy target and the hybridization efficiencies were similar. The means (±SD) of p40 and c-myc signals in the BPH samples were 2.2±0.3 and 2.1±0.2, respectively.

Fluorescent images were captured with Zeiss Axioplan 2 microscope (Carl Zeiss Jena GmbH, Jena, Germany) equipped with Hamamatsu C9585 camera (Hamamatsu Photonics, K.K., Japan) and ISIS software program (Metasystems GmbH, Altslusheim, Germany). Tumors that showed more than 20% of nuclei with an increased copy number of either eIF3-p40 or c-myc were considered to have amplification. In the cases with amplification, the level of amplification was determined counting only nuclei with an increased number of signals.

The tumors were classified into three groups: normal (no increase in the eIF3-p40 or c-myc copy number), low-level amplification (3 to 5 copies per cell) and high-level amplification (≧5 copies of the genes per cell or a gene/centromere ratio>2) (FIGS. 1A and 1D). High-level amplification of eIF3-p40 was found in SK-Br-3, MDA436, MCF-7, and PC-3, in accordance with the gain of 8q found by CGH in these cell lines (FIG. 2).

### EXAMPLE b 3

Amplification of eIF3-p40 in Breast Cancer and Prostate Cancer Samples

To show that eIF3-p40 is amplified in native tumors obtained from patients suffering from breast or prostate cancer, 44 hormone-refractory locally recurrent prostate carcinomas and 39 untreated primary breast carcinomas were screened for the eIF3-p40 copy number by FISH essentially as described in Example 2.

Of the prostate carcinomas, 30% (13/44) showed high-level amplification of eIF3-p40, whereas the remaining cases showed a low-level copy number gain (3 to 4 copies) of eIF3-p40. In prostate tumors with high-level amplification, the mean (±SD) copy number of eIF3-p40 was 6.7 (±1.5). Of the breast cancers, 18% (7/39) showed high-level amplification of eIF3-p40, 43% (17/39) showed a low-level gain, while the remaining tumors (39%) showed two copies of eIF3-p40. The mean copy number of eIF3-p40 was 8.5 (±2.9) in the breast tumors with high-level gene amplification.

Additionally, 19 selected breast carcinomas with high-level c-myc amplification demonstrated by Southern blot (Borg et al., lnt J Cancer, 9, 687-91, 1992) were analyzed. Sixteen of the tumors displayed high-level amplification of eIF3-p40 with a mean copy number of 21.8 (±21.12).

### EXAMPLE 4

Comparison of the Amplification of eIF3-p40 and the c-myc Oncogene in Breast Cancer and Prostate Cancer Samples

Because eIF3-p40 is localized close to the c-myc oncogene, the copy number of c-myc was also studied by FISH as described in Examples 2 and 3. The results are shown in FIG. 5.

In breast and prostate cancer cell lines, the copy numbers of c-myc and eIF3-p40 were identical, except in PC-3, where eIF3-p40 was present in 15 copies and c-myc only in nine copies. All unselected hormone-refractory prostate carcinomas showed equal copy numbers of eIF3-p40 and c-myc, whereas one of the unselected breast carcinomas showed high-level amplification of c-myc, but only low-level amplification of eIF3-p40. Of the selected 20 breast carcinomas, three showed about five times higher copy number of eIF3-p40 than c-myc, whereas one case showed about twice as many c-myc signals as eIF3-p40 signals.

### EXAMPLE 5

Association of the Amplification of eIF3-p40 With the Over-expression of the Gene

The amplification of the putative target proto-oncogenes is thought to lead to their over-expression. The expression levels of eIF3-p40 and c-myc in breast cancer cell lines MDA436, MCF-7 and SK-Br3 and in prostate cancer cell line PC-3 were compared using the Northern analysis. The Northern blot analysis was performed as described in Example 1. The expression levels were quantitated using Phosphoimager (Molecular Dynamics Inc, Sunnyvale, Calif., USA). The expression of β-actin was used to control the loading differences. The results of the Northern blot analysis are shown in FIG. 2.

The expression of eIF3-p40 was also examined in breast and prostate tumors using semi-quantitative mRNA in situ hybridization. mRNA in situ hybridization was performed using a 780 bp EcoRI-HincII-fragment from cDNA EST-clone 595376 (GenBank accession no. AA173710), which was subcloned in pBluescript SK vector (Stratagene, La Jolla, SA, USA) and used for in vitro transcription of eIF3-p40 antisense and sense riboprobes. A cytokeratin antisense probe, derived from a 690 bp EcoRI-SmaI fragment of EST-clone 487868 (Genbank accession no. AA044589) was used to control the quality of RNA of the samples. Hybridization was carried out on 27 formalin-fixed paraffin-embedded hormone-refractory prostate carcinomas, 34 primary breast carcinomas, 1 normal breast tissue, and 3 benign prostate hyperplasias (BPHs) with ³³P-dUTP-labeled riboprobes. In addition, six BPH lesions adjacent to the carcinoma were analyzed. The hybridized sections were exposed to Amersham β-max Hyperfilms for three days, whereafter the slides were developed and scanned using Personal Densitometer SI (Molecular Dynamics Inc.). The expression levels were quantitated with ImageQuaNT software program (Molecular Dynamics Inc.) using the volume quantitation option. The first representative equal-sized objects were selected from each slide. The quantitation results were given as integrated intensity of all pixels in the objects excluding the background. For microscopic examination, the sections were immersed in autoradiographic emulsion NTB2 (Kodak) and exposed for 4 weeks at ±4° C. After the autogradiographic signals, the sections were counterstained with hematoxylin and examined in Nikon Microphot-SA (Nikon Corp., Tokyo, Japan) microscope equipped with an epipolarization filter.

The results of the Northern blot analysis indicate an increased expression of eIF3-p40 in the MDA436, MCF-7, SK-Br3, and PC-3 cell lines that show high-level amplification of eIF3-p40 by FISH, as compared to the expression level in the ZR75-1 (FIG. 2). The expression levels of c-myc show clearly less variation than eIF3-p40 expression. The relative level of expression of the genes is given in proportion to the expression in ZR75-1. The relative copy number (gene vs. centromere copy number) of eIF3-p40 and c-myc are also shown.

The expression of eIF3-p40 was related to its gene copy number, whereas there was no clear association between the expression and the amplification status of c-myc (FIG. 2).

The expression of eIF3-p40 was also examined in prostate and breast tumors with semi-quantitative mRNA in situ hybridization (FIG. 3). The hormone-refractory prostate carcinomas expressed over four times more eIF3-p40 than benign prostate hyperplasia (BPH) tissues (FIG. 4) (Mann-Whitney U-test; p=0.0021). The level of the eIF3-p40expression was higher in breast carcinomas with high-level amplification than in breast carcinomas with low-level or no amplification (Kruskal-Wallis test; p=0.028). Thus, over-expression of the eIF3-p40 gene was significantly associated with its amplification, suggesting that it is one of the putative target genes amplified in the 8q23-q24 region. The expression of eIF3-p40 was higher in hormone-refractory prostate carcinomas than in BPH (p=0.002). Similarly, the expression of eIF3-p40 was higher in breast carcinomas with high-level amplification than in breast carcinomas with low-level amplification (p=0.029) of the eIF3-p40 gene.

### EXAMPLE 6

Production of Recombinant eIF3-p40 Protein and Generation of Monoclonal Antibodies (MAbs) Against the eIF3-p40 Protein

The coding sequence of the eIF3-p40 gene (sequence id. no. 3), which originated from EST 346021 (Accession no. W72146), was subcloned to a pTrcHis vector according to the manufacturer's instructions (Invitrogen Corp., Carlsbad, Calif., USA). The histidine-tagged recombinant protein was produced in Escherichia coli and purified with Xpress Protein Purification System (Invitrogen Corp Carlsbad, Calif., USA.) In a native form according to the manufacturer's instructions.

Five mice (Balb/c line) were immunized intraperitoneally with the recombinant p40 protein (25 μg/mouse) in Complete Freund's Adjuvant. Forty days later, the animals were boosted intramuscularly by the same antigen (35 μg/mouse) in Incomplete Freund's Adjuvant. After eight days, mouse antisera were taken and screened for the specific antibodies using the ELISA technique with a homologous antigen. Titers of 1:500-1:1000 were found. Seven days later all the mice were immunized intravenously by the same antigen with a dose of 40 μg/mouse. Four days later, the splenocytes from one mouse were fused with the Sp/2 myeloma cell line (a Balb/c mouse line), whereas the rest of the animals were boosted intravenously with the antigen (40 μg/mouse) every three weeks. Four days after each boost the splenocytes from one mouse were fused with the Sp/2 myeloma cell line. Thus, three more fusions were done. The mouse thymocytes were used as feeder cells as well as for recloning of hybridomas.

Five monoclonal antibodies (mabs) were produced: P40 2.1 (IgG2a-glass), P40 3.1 (not typed), P40 4.1 (IgG1), P40 5.1 (not typed), and P40 6.1 (IgG3). All the antibodies were specific to the recombinant p40 antigen by the ELISA and Western immunoblotting techniques. Heterologous recombinant proteins produced in the same vector (pTrcHis Vector) and host (E. coli) were used as negative controls.

The specific reactivity of MAbs P40 2.1, P40 3.1, and P40 4.1 was tested using Western immunoblotting with native proteins derived from the epithelial cell lines ZR-75-1 and SK-Br-3. MAb P40 3.1, which was found to be specific to the recombinant antigen eIF3-p40, showed two strong 15 kDa (major) and 21 kDa (minor) bands in Western immunoblotting of native cellular proteins derived from SK-Br-3 and ZR75-1 cells lines and blood cells (data not shown). The nature of the “specific” native proteins remains to be defined. MAbs p4 6.1 and 4.1 regocnized a 40 kDA band corresponding to the size of the eIF3-p40 protein (FIG. 6). On the other hand, P40 5.1, which was also specific to the recombinant antigen p40, failed to detect a corresponding native protein in Western immunoblotting.

1. A method for diagnosing aggressive forms of carcinomas, characterized by detecting the presence or absence of amplification and/or expression of p40 subunit of eukaryotic translation initiation factor 3 or a functional variant or functional fragment thereof in a biological sample.

2. A method of claim 1, characterized in that the carcinoma is a breast carcinoma or a prostate carcinoma.

3. A method of claim 1 or 2, characterized in that the detection is performed using a gene-technological method.

4. A method of claim 3, characterized in that the detection is performed using an in situ hybridization method, such as fluorescence in situ hybridization (FISH) or mRNA in situ hybridization, a Southern analysis, an RT-PCR, and a Northern analysis.

5. A method of claim 1 or 2, characterized in that the detection is performed using an immunological method, such as a Western analysis, immunochistology and an immunoassay.

6. A method of claim 5, charactarized in that the detection is performed using immunochistology.

7. Use of p40 subunit of eukaryotic translation initiation factor 3 or a functional variant thereof or a functional fragment thereof for the diagnosis of aggressive forms of carcinomas.

8. Use of claim 7, characterized in that the carcinoma is a breast or a prostate carcinoma.

9. A method for identifying cancer patients suffering from an aggressive form of carcinoma, such as breast and prostate cancer, characterized by detecting the presence or absence of p40 subunit of eukaryotic translation initiation factor 3 or a functional variant or functional fragment thereof.

10. Use of p40 subunit of eukaryotic translation initiation factor 3 or a functional variant or functional fragment thereof for the identification of patients suffering from aggressive forms of carcinomas, such as breast cancer and prostate cancer.

11. A method for evaluating the aggressivity of carcinomas, characterized by determining the presence or absence of p40 subunit of eukaryotic translation initiation factor 3 or a functional variant or functional fragment thereof in a tumor sample.

12. Use of p40 subunit of eukaryotic translation initiation factor 3 or a functional variant or functional fragment thereof as a diagnostic agent.

13. Use of p40 subunit of eukaryotic translation initiation factor 3 or a functional variant or fragment or mutated form thereof in therapy.

14. A diagnostic kit for the identification of p40 subunit of eukaryotic translation initiation factor 3 or its gene product, in a biological sample, characterized by containing a monoclonal antibody capable of reacting specifically with p40 subunit of eukaryotic translation initiation factor 3 or a gene product thereof, and reagents necessary for visualization or quantification.

15. A diagnostic kit of claim 14, characterized in that at least one of said reagents necessary for visualization or quantification is an agent capable of detecting said monoclonal antibody.

16. A diagnostic kit for the detection of specific antibodies against p40 subunit of eukaryotic translation initiation factor 3 or a functional variant or functional fragment thereof in a biological sample, characterized by containing p40 subunit of eukaryotic translation initiation factor 3 or a functional variant or functional fragment thereof and suitable reagents necessary for the detection.
